(19) United States
(12) Patent Application Publication
(10) Pub. No.: US 2026/0098286 A1
(43) Pub. Date: Apr. 9, 2026

(54) Low Dose Psilocybin in Foodstuff and Microbes for Same

(71) Applicant: ATX PHARMERS LLC, Austin, TX (US)

(72) Inventors: Vivek PANDYA, Austin, TX (US); Tamsen VALOIR, Houston, TX (US)

(73) Assignee: ATX PHARMERS LLC, Austin, TX (US)

(21) Appl. No.: 19/416,712

(22) Filed: Dec. 11, 2025

Related U.S. Application Data
(62) Division of application No. 17/876,134, filed on Jul. 28, 2022.
(60) Provisional application No. 63/228,853, filed on Aug. 3, 2021.

(51) Int. Cl. C12P 17/10; C12N 1/16; C12N 1/20; C12N 9/02; C12N 9/10; C12N 9/12; C12N 9/88; C12N 15/52; C12N 15/74; C12N 15/81

(52) U.S. Cl. CPC C12P 17/10; C12N 1/16; C12N 1/20; C12N 9/0083; C12N 9/1007; C12N 9/1205; C12N 9/88; C12N 15/52; C12N 15/74; C12N 15/81; C12N 2830/002; C12Y 114/99; C12Y 201/01; C12Y 207/01; C12Y 401/01

(57) ABSTRACT

Microbes are transformed with psilocybin genes under the control of weak or medium level promoter to make low levels of psilocybin therein. Low dose, microdose and sub-microdose foodstuff are then made with such microbes.

### PRIOR RELATED APPLICATIONS

This application is a Divisional Application of U.S. application Ser. No. 17/876,134, filed Jul. 28, 2022, which claims priority to U.S. Ser. No. 63/228,853, filed Aug. 3, 2021. The entire contents of such prior applications are incorporated by reference in its entirety for all purposes.

### FEDERALLY SPONSORED RESEARCH STATEMENT

Not applicable.

### FIELD OF THE DISCLOSURE

The disclosure generally relates to the production of foodstuffs containing reliably low levels of psilocybin.

### BACKGROUND OF THE DISCLOSURE

Psilocybin (4-phosphoryloxy-N,N-dimethyltryptamine) is a naturally occurring, tryptamine-derived psychedelic prodrug compound produced by more than 200 species of fungi, colloquially known as “magic mushrooms.” The most potent are members of the genus Psilocybe, such as P. azurescens, P. semilanceata, and P. cyanescens, but psilocybin has also been isolated from about a dozen other genera.

Psilocybin has a low toxicity and a low harm potential, but in spite of this, possession of psilocybin-containing mushrooms has been outlawed in most countries, and it has been classified as a schedule 1 drug in the US. Schedule I drugs are defined as drugs with a high potential for abuse or drugs that have no recognized medical uses. However, psilocybin mushrooms have had numerous medicinal and religious uses in dozens of cultures throughout history and have a significantly lower potential for abuse than other Schedule I drugs.

Recognizing the safety profile of these compounds and leaving the war of drugs behind as bad policy, many US cites and some states have already decriminalized the use of magic mushrooms, including Denver, Colorado, Oakland, California, Santa Cruz, California, and Ann Arbor, Michigan. More decriminalization is expected, and even outright legalization, and this will open up the market for recreational use and products containing psilocybin and psilocin and related molecules.

As a prodrug, psilocybin is quickly converted by the body to psilocin, which has mind-altering effects similar, in some aspects, to those of LSD, mescaline, and DMT. In general, the effects include euphoria, visual and mental hallucinations, changes in perception, a distorted sense of time, and perceived spiritual experiences. It can also include possible adverse reactions such as nausea and panic attacks. Psilocin is structurally similar to human signaling molecules such as serotonin, and has been shown to bind to over 15 human serotonin-related receptors.

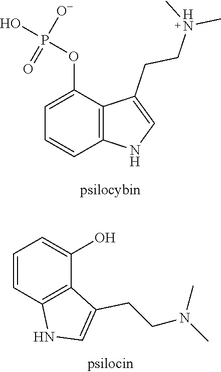

See also FIG. 1A-B for these two compounds.

Unfortunately, the content of psilocybin and psilocin in hallucinogenic mushrooms is too low (0.2%-1% dry weight) to make extraction a commercially viable option, and chemical synthesis is complicated and expensive. Psilocin can be obtained by dephosphorylation of natural psilocybin under strongly acidic or under alkaline conditions (hydrolysis). Another synthetic route uses the Speeter-Anthony tryptamine synthesis starting from 4-hydroxyindole. However, psilocin is relatively unstable in solution due to its phenolic hydroxy (—OH) group. In the presence of oxygen it readily forms bluish and dark black degradation products. Similar products are also formed under acidic conditions in the presence of oxygen and Fe³⁺ ions (Keller's reagent).

Because of the difficulty in synthesizing these compounds and their poor stability, there have been efforts to clone the genes for the pathway into other species. US20210147888, for example, describes insertion of the genes into yeast, such as fungal species comprising: Schizosaccharomyces cerevisiae, Schizosaccharomyces japonicus, Schizosaccharomyces pombe, Schizosaccharomyces cryophilus, Saccharomyces cerevisiae, Kluyveromyces lactis, Kluyveromyces dobzhanskii, and Yarrowia lipolytica. With the addition of L-tryptophan to the growing culture medium, these yeast were able to produce psilocybin. A psilocybin titer was not given, but the inventors attempted to produce as large an amount as possible since their interest lay in producing these compounds for purification and pharmaceutical use.

WO2021067626 describes modulating the psilocybin biosynthesis pathway in fungi or other organisms, and WO2021052989 describes the production of 4-hydroxytryptamine and other halogenated tryptophan derivatives thereof in a yeast cell, such as S. cerevisiae, S. kluyveri, S. bayanus, S. exiguus, S. sevazzi, S. uvarum, S. boulardii, Kluyveromyces, lactis, K. marxianus var. marxianus, K. thermotolerans, Candida, utilis, C. tropicalis, C. albicans, C. lipolytica, C. versatilis, Pichia stipidis, P. pastohs, P. sorbitophila, Cryptococcus aerius, Debaromyces hansenii, Hansenula, Pichia, pastoris, Yarrowia lipolytica, Zygosaccharomyces bailii, Torulaspora delbrueckii, Schizosaccharomyces pombe, Brettanomyces bruxellensis, Penicillium, Rhizopus, Fusarium, Fusidium, Gibberella, Mucor, Mortierella, and Trichoderma.

WO2021086513 describes the same sort of modification to prokaryotes, such as Escherichia coli, Corynebacterium glutamicum, Vibrio natriegens, Bacillus subtilis, Bacillus megaterium, Escherichia coli Nissle 1917, Clostridium acetobutlyicum, Streptomyces coelicolor, Lactococcus lactis, Pseudomonas putida, Streptomyces clavuligerus, and Streptomyces venezuelae.

However, each of these patents is concerned with pharmaceutical uses, and thus each strives to maximize production and isolation of product. As such, any food products containing same would likely be too strong and unsafe for consumer consumption.

Thus, what is needed in the art are beer, bread and wine yeast, Lactobacillus and other generally recognized as safe (GRAS) microbes able to make modest and controllable amounts of psilocybin and related compounds, such that, e.g., beer, wine and other foods and beverages could provide reliable, safe, low doses, without negatively impacting the flavor profile of the foodstuff.

### SUMMARY OF THE DISCLOSURE

This invention provides beer, bread and wine yeast, Lactobacillus and other microbes with low level transcription of the genes required to produce psilocybin in low and predictable amounts, such that consumables can be made therefrom, and the dosage will be low enough for safe use. Preferably, the dosage is a microdose, but even low doses, or sub-microdoses, may be possible as well.

The effects of the psilocybin begin 10-40 minutes after ingestion, and last 2-6 hours depending on dose, species, and individual metabolism. The half-life of psilocybin is 163±64 minutes when taken orally. Psilocybin is converted in the liver to the pharmacologically active psilocin, which is then either glucuronated to be excreted in the urine or further converted to various psilocin metabolites. See FIG. 2.

As psilocybin is converted to psilocin, it undergoes a first-pass effect, whereby its concentration is greatly reduced before it reaches the systemic circulation. Psilocin is broken down by the enzyme monoamine oxidase to produce several metabolites that can circulate in the blood plasma, including 4-hydroxyindole-3-acetaldehyde, 4-hydroxytryptophol, and 4-hydroxyindole-3-acetic acid. Some psilocin is not broken down by enzymes and instead forms a glucuronide; this is a biochemical mechanism animals use to eliminate toxic substances by linking them with glucuronic acid, which can then be excreted in the urine.

Psilocin is glucuronated by the glucuronosyltransferase enzymes UGT1A9 in the liver, and by UGT1A10 in the small intestine. Based on animal studies, about 50% of ingested psilocybin is absorbed through the stomach and intestine. Within 24 hours, about 65% of the absorbed psilocybin is excreted into the urine, and a further 15-20% is excreted in the bile and feces. Although most of the remaining drug is eliminated in this way within 8 hours, it is still detectable in the urine after 7 days.

Clinical studies show that psilocin concentrations in the plasma of adults average about 8 μg/liter within 2 hours after ingestion of a single 15 mg oral psilocybin dose; psychological effects occur with a blood plasma concentration of 4-6 μg/liter. Psilocybin is about 100 times less potent than LSD on a weight per weight basis, and the physiological effects last about half as long.

As used herein, the dosage levels per unit of consumable food or drink are as follows:

Microdose: 50-250 micrograms (μg) of pure psilocybin—here the dose is so low that there are no psychedelic effects. This means the consumer will not notice any changes in perception, such as visual or auditory effects, but may experience some changes in thinking, concentration, higher levels of creativity, relief from depression, less anxiety, emotional openness, relief from menstrual pain, heightened spiritual awareness and more energy.

Low Dose: >250-1000 μg—Here the consumer may experience a slight change in visual perception, a slight body high, a feeling of euphoria.

Monoamine oxidase inhibitors (MAOI) have been known to prolong and enhance the effects of DMT and one study assumed that the effect on psilocybin would be similar since it is a structural analogue of DMT. Alcohol consumption may enhance the effects of psilocybin, because acetaldehyde, one of the primary breakdown metabolites of consumed alcohol, reacts with biogenic amines present in the body to produce MAOIs related to tetrahydroisoquinoline and β-carboline. Thus, an even lower dose than a microdose (a sub-microdose) may be suitable for beer and wine use, and we anticipate that 0.75, 0.5, or 0.25 of a microdose per unit of alcoholic beverage may be more suitable than a full microdose. Suitable sub-microdoses might be 200 μg/340 ml beer, 150 μg/340 ml beer, or even 100, 75, or 50 μg/340 ml beer, and a microdose of 250-1000 μg/340 ml beer.

Preferably, the foodstuff is tested, and in the event of variation, a concentration adjustment (e.g., dilution) is made to provide the desired low dose, microdose, or sub-microdose levels.

The genetic source organisms can include, but are not limited to: Psilocybe cubensis, Psilocybe cyanescens, Panaeolus cynascens, Gymnopilus junonius, and Gymnopilus dilepis. DNA sequences are synthesized and cloned using techniques known in the art, or preferably are transferred from preexisting clones, since many genes have already been cloned.

Gene expression can be controlled by inducible or constitutive promoter systems using the appropriate expression vectors, but are preferably constitutive weak or medium promotors producing reliable low dose levels of drug without adverse effects on flavoring.

“Weak”, “strong” and “medium” promoters are terms of art and most commercial suppliers of promoters classify promoters as such, but without providing any numerical cutoffs since expression levels are protein and condition dependant. Herein, we use the supplier or literature description of a promoter as medium or weak, even though actual binding and transcription levels may vary. As a rough guide however, a strong promoter will be at least 100 fold more active than a weak promoter, and at least 10 fold more active that a medium one. A list of yeast promoters can be found at parts.igem.org/Yeast.

Intermediates or side products of psilocybin synthesis include norbaeocystin, baeocystin, 4-hydroxytryptophan, 4-hydroxytryptamine, aeruginascin, psilocin, norpsilocin, or 4-hydroxy-N,N,N-trimethyltryptamonium (4-OH-TMT). In some embodiments the intermediate of psilocybin is norbaeocystin, baeocystin, 4-hydroxytryptophan, or 4-hydroxytryptamine. In some embodiments, the side product of psilocybin is aeruginascin, psilocin, norpsilocin, or 4-hydroxy-N,N,N-trimethyltryptamonium (4-OH-TMT).

The invention includes any one or more of the following embodiment(s) in any combination(s) thereof, but each possible combination is not separately listed in the interest of brevity.

A recombinant microbe, a) the microbe being of a species generally recognized as safe (GRAS) for human consumption; b) the microbe expressing each of a PsiD gene, a PsiH gene, a PsiK gene, and a PsiM gene, each gene under the control of a weak promoter or a medium promoter; c) the microbe producing psilocybin or a related compound.

Any microbe herein described, wherein the microbe has native genes for the production of tryptophan, preferably one or more of the native genes being upregulated.

Any microbe herein described, wherein each promoter is a constitutive promoter.

Any microbe herein described, wherein one or more or each promoter is an inducible promoter or a constitutive promoter, or one or more promoters is a weak constitutive promoter or one or more promoters is a medium constitutive promoter.

Any microbe herein described, wherein the microbe is Saccharomyces, Lactobacillus or Brettanomyces.

A foodstuff made with any microbe herein described, wherein the foodstuff has <10 mg or preferably <1 mg of psilocybin per serving of the foodstuff, or <250 μg per serving of the foodstuff or even <100 μg per serving of the foodstuff.

A foodstuff made with any microbe herein described, the foodstuff being an alcoholic beverage, such as e.g., beer, wine, cider, sake, or mead. One particularly preferred foodstuff is beer.

A foodstuff made with a recombinant GRAS microbe that produces psilocybin, wherein the foodstuff has <10 mg or preferably <1 mg of psilocybin per serving of the foodstuff or even <250 μg or <100 μg of psilocybin per serving of the foodstuff.

Any percentage herein is a w/v percentage basis for fluids and w/w basis for solids, unless noted otherwise.

As used herein psilocybin levels are preferably measured by high performance liquid chromatographic procedures with column-switching coupled with electrochemical detection (HPLC-ECD).

In a foodstuff, the levels are ideally quite reliable, at +/−10% or preferably +/−5% of the stated levels, or even ±3%, ±2%, or ±1%.

By “consumable foodstuff” or just “foodstuffs” herein, we mean to include both food and beverages.

By “microdose” we mean 250-1000 μg of active ingredient in a single serving. A “sub-microdose” is <250 μg/serving. In foods that are frequently overconsumed (e.g., multiple servings, such as beer), the dose may be lower.

“Serving size” or “serving” is a definition developed by the United States Department of Agriculture—the serving size defines a specific amount of a particular food that represents the recommended portion for one sitting. The serving size creates a standard reference used on food labels. The relevant serving size is as defined at the time of infringement and will typically be printed on the labels.

The use of the word “a” or “an” in the claims or the specification means one or more than one, unless the context dictates otherwise.

The term “about” means the stated value plus or minus the typical margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim. The phrase “consisting of” is closed, and excludes all additional elements. The phrase “consisting essentially of” excludes additional material elements, but allows the inclusions of non-material elements that do not substantially change the nature of the invention, such as instructions for use, packaging, flavorants, preservatives, and the like. Any claim or claim element introduced with the open transition term “comprising,” may also be narrowed to use the phrases “consisting essentially of” or “consisting of,” and vice versa. However, the entirety of claim language is not repeated verbatim in the interest of brevity herein.

The following abbreviations are used herein:

| ABBREVIATION | TERM |
|---|---|
| GMO | Genetically modified organism |
| GRAS | Generally recognized as safe |
| LSD | Lysergic acid diethylamide |
| DMT | N,N-Dimethyltryptamine |
| MAOI | Monoamine oxidase inhibitor |
| PSB | Psilocybin |
| PSC | Psilocin |
| THCA | Tetrahydrocannabinolic acid |

### BRIEF DESCRIPTION OF THE DRAWINGS

FIG. 1A. Psilocin properties.

FIG. 1B. Psilocybin properties.

FIG. 2. Psilocybin metabolism.

FIG. 3. Conversion of tryptophan to psilocybin.

FIG. 4. Tryptophan pathway in yeast.

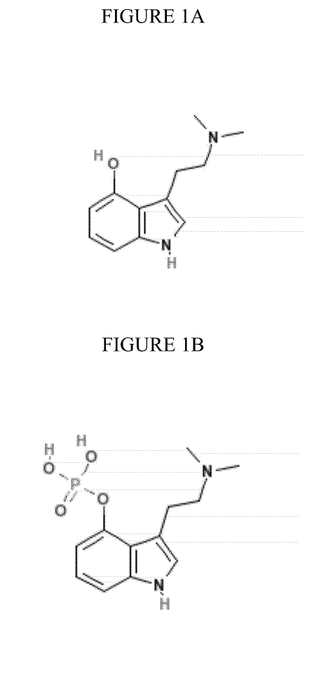

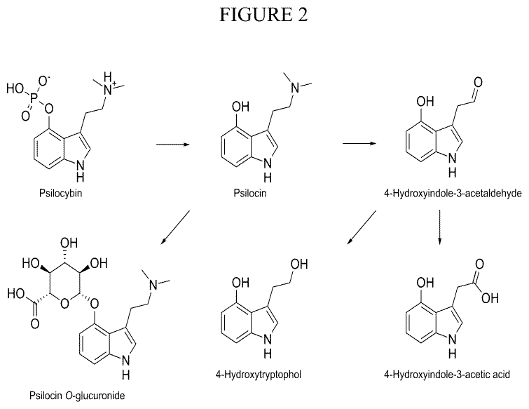

### DETAILED DESCRIPTION

The disclosure is exemplified with respect to brewer's yeast and beer made therewith, but it not intended to be so limited, and the invention can be applied to any microbe used in food production and the resulting foodstuffs. Thus, wine, bread and beer yeasts, which are mostly Saccharomyces species are included, as well as Pichia, Torulaspora, Lactobacillus, Brettanomyces, and the like. Additional microbes that have been generally recognized as safe by the FDA are found in 21 CFR 184, incorporated by reference in its entirety for all purposes.

The disclosure provides novel consumable foodstuffs containing low doses, microdoses, or sub-microdoses of psilocybin and/or related compounds. Beer, bread and wine yeast Lactobacillus, and other GRAS microbes that synthesize low levels of psilocybin are also provided, such that foodstuff with low dose, microdose or sub-microdose levels can be made therewith.

The correlation between the level of psilocybin production in e.g., brewer's yeast, and the resulting psilocybin levels in beer is not yet known and will need to be elucidated. However, since the yeast remain in an unpasteurized and unfiltered beer, whatever is made will remain in the beer (excepting harvesting and re-pitching the yeast and excepting any drug lost due to degradation). Further, different brewing techniques will vary the levels therein. Thus, one of our first experiments will be to assess the carry-over of the drug from the yeast to the final beer product, for example as described in Spevacek 2016. Ultimately, both top and bottom yeasts will need to be tested, along with a variety of beer recipes to fully appreciate this relationship, however, proof of concept work may use a single methodology.

In the interim, we can look to similar experiments done with cannabis producing yeast, where scientist were able to produce 8 mg/liter of THCA in yeast using the strong inducible promoters pGal1 and pGal10, but yields need to increase 100 fold to make production competitive with the plant. Here, we have little need for higher production, and strong promoters are probably not needed. Instead a lower level constitutive promoter could be used, depending on yeast reaction to this metabolite. If the yeast react badly to lower levels of psilocybin (e.g., demonstrating poor growth), an inducible promoter could be used, but we expect that titrating a constitutive low level of psilocybin production will be easier and more compatible with beer and other foodstuff production. Thus, weak or medium constitutive promoters are expected to be preferred.

### PROOF OF CONCEPT

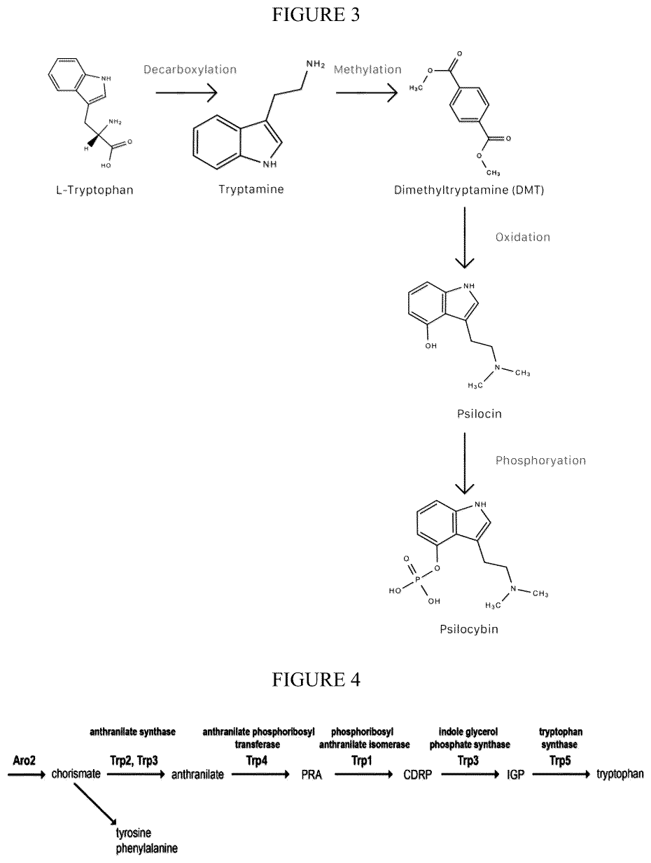

Proof of concept experiments will be performed in a Saccharomyces yeast. The pathway from tryptophan to psilocybin is shown in FIG. 3 and the genes have already been cloned from multiple species and the enzymes are EC 4.1.1.105; EC 2.7.1.222; EC 1.14.99.59; and EC 2.1.1.345. Since we desire to make low levels of psilocybin, we anticipate using medium or even weak promoters and will test both constitutive as well as inducible promoters.

The genes and the GenBank accession numbers needed to put a psilocybin synthesis pathway into yeast are listed in Table 1, but these cloning experiments are not detailed as that work has already been done. We hope to obtain starting materials from another lab, e.g., the Keasling, Borodina or another laboratory:

TABLE 1
Psilocybin Gene/proteins

| | | Acc. No. | Acc. No. | |
|---|---|---|---|---|
| Gene | Enzyme | gene | prot | Species |
| PSID | L-tryptophan | KY984101.1 | ASU62239.1 | P. cubensis |
| | decarboxylase | | | |
| PSID | L-tryptophan | KY984104.1 | ASU62242.1 | P. cyanescens |
| | carboxylase | | | |
| PSIH | Tryptamine-4- | MF000993.1 | ASU62246.1 | P. cubensis |
| | monooxygenase | | | |
| PSIH | Tryptamine-4- | MF000997.1 | ASU62250.1 | P. cyanescens |
| | monooxygenase | | | |
| PSIK | 4-hydroxytrypta- | KY984099.1 | ASU62237.1 | P. cubensis |
| | mine kinase | | | |
| PSIK | 4-hydroxytrypta- | KY984102.1 | ASU62240.1 | P. cyanescens |
| | mine kinase | | | |
| PSIM | norbaeocystin | KY984100.1 | ASU62238.1 | P. cubensis |
| | methyltransferase | | | |
| PSIM | norbaeocystin | KY984103.1 | ASU62241.1 | P. cyanescens |
| | methyltransferase | | | |

The requisite strong promoter PSID-H-K-M clones will be obtained from a lab and the clones reengineered to have medium and weak promotors. Sigma-Aldrich makes promotor sets for just this kind of testing. The tested promoters may thus include:

TABLE 2
Yeast promoters from Sigma-Aldrich

| Catalog | |
|---|---|
| No. | Promoter |
| OGS535 | PSF-STE5-URA3-WEAK PROMOTER YEAST PLASMID, |
| | plasmid vector for molecular cloning |
| OGS537 | PSF-ADH1-URA3-MEDIUM STRENGTH YEAST |
| | PROMOTER PLASMID, plasmid vector for molecular |
| | cloning |
| OGS536 | PSF-GAL1-URA3-GALACTOSE INDUCIBLE YEAST |
| | PLASMID, plasmid vector for molecular cloning |

Additional yeast promoters that could be tested include pSED1, pHXT7, pPDC1, pTEF1, pTPII (medium-high expression in aerobic cultivation, and moderate expression in microaerobic fermentation); pTEF2 (moderate expression in aerobic culture and weak expression in microaerobic fermentation); pZWF1 and pSOL4 (moderate expression in aerobic cultivation, while showing weak in microaerobic fermentation); and pALD3 and pTKL2 (moderate promoter activity in aerobic cultivation, but showed almost no activity in microaerobic fermentation).

In addition to testing gene expression using the above promoters, the strong inducible promoter in the original clones will also be tested at varying levels of inducement. We do not anticipate using an inducible promoter since the ultimate cultured foodstuff will be beer and minimal impact on flavor profiles is desirable, and many inducers may not be consistent with this goal. Nevertheless, these experiments will help us to characterize production levels under different levels of gene expression and will be of value in selecting the ultimate promoters for use in making GRAS microbes and foodstuffs from same.

Yeast growth curves in culture for each strain of yeast will be determined, and the level of psilocybin also measured. Beer will be made with one or more of these yeasts, and psilocybin levels again determined, and levels adjusted as needed with non-psilocybin beer. Beer quality overall will be assessed, and the effects of varying levels of psilocybin on flavor and crispness determined. In some embodiments, psilocybin-yeast may be combined with non-psilocybin yeasts in order to modify the flavor profile.

From these data we hope to obtain at least a rough correlation between levels of psilocybin in the yeast, and final levels in unpasteurized and unfiltered beers. The promoters and conditions that produce about 250-1000 μg psilocybin/340 ml beer will be chosen for future studies.

Depending on the levels of psilocybin and related compound in both the microorganism and in the resulting foodstuff, it may be helpful to upregulate the genes required for the synthesis of tryptophan—the starting material for the pathway. The yeast (Saccharomyces) TRP genes are listed in Table 3 and the pathway shown in FIG. 4 where PRA=N-(5′-phosphoribosyl)-anthranilate, CDRP=1-(o carboxyphenylamino)-1′-deoxyribulose-5′ phosphate, IGP=indole glycerol phosphate.

TABLE 3
Saccharomyces tryptophan enzymes

| Gene | | UniProt |
|---|---|---|
| name | Enyzme | Acc. No |
| ARO2 | Chorismate synthase | P28777 |
| TRP1 | N-(5′-phosphoribosyl)anthranilate isomerase | P00912 |
| TRP2 | Anthranilate synthase component 1 | P00899 |
| TRP3 | Multifunctional protein, Component I catalyzes the | P00937 |
| | formation of anthranilate using ammonia rather than | |
| | glutamine, whereas component II provides glutamine | |
| | amidotransferase activity. | |
| TRP4 | Anthranilate phosphoribosyltransferase | P07285 |
| TRP5 | Tryptophan synthase | P00931 |

We anticipate that obtaining reliably low dose, microdose, or sub-microdose levels of psilocybin in a foodstuff such as wine or beer may be difficult as the behavior of microbes in culture is quite variable with varying conditions. However, if the level of the microbe is set such that slightly higher dosages are reliably obtained, it will also be possible to dilute e.g., the beer with a non-psilocybin containing beer of the same style, thus providing the consumer with a reliable product. Likewise, the wines can be blended as is commonly done today. As another option, it may be possible to blend microbes, e.g., psilocybin-containing yeast with non-GMO lactobacillus, thereby producing a sour beer. In all cases, reliability of levels will be important to the consumer, and we expect to aim for psilocybin levels within ±10% but preferably ±5% of the stated levels in all cases.

It is anticipated that stability tests will be required to confirm that the storage of the foodstuff does not adversely affect the levels of psilocybin and related compounds. If there is degradation, packaging, presentation and instructions for use will be adjusted accordingly to minimize loss. It is known, however, that these compounds are not stable in light and air, thus airtight packaging, possible under nitrogen blankets, in opaque foil, aluminum cans, covered glass or dark glass will be used.

Although we have discussed beer as one possible foodstuff, obviously other possible alcoholic beverages can be included herein, such as wine, mead, cider, ayran, tella, borde, shamita, korefe, keribo, cheka, tej, algol, ikigage, oti-oka, kwete, busaa, makgeolli, pulque, and sake, to name a few. There are also many nonalcoholic food stuffs, such as kombucha, yogurt, sauerkraut, kefir, kimchi, miso, pickles, tempeh, natto, sourdough, olives, cheese, to name just a few. In addition, a cultured food product can be further treated, such as in distilling sprits from alcoholic beverages.

However, we anticipate that the earliest markets to be developed for the microbes described herein will be in beer since the craft beer market is already vast and craft brewers (and beer drinkers) are quite adventurous.

The following references are incorporated by reference in their entirety for all purposes.
Spevacek, A. R.; Benson, K. H.; Bamforth, C. W.; Slupsky, C. M. “Beer metabolomics: molecular details of the brewing process and the differential effects of late and dry hopping on yeast purine metabolism.” J. Inst. Brewing, 122(1): 21-28 (2016), available online at onlinelibrary.wiley.com/doi/full/10.1002/jib.291.
Milne N.; Thomsen, P.; Knudsen, M.; Kristensen, M.; Borodina, I. “Metabolic engineering of Saccharomyces cerevisiae for the de novo production of psilocybin and related tryptamine derivatives.” Metab Eng. 2020 July; 60:25-36, available online at ncbi.nlm.nih.gov/pmc/articles/PMC7232020/
Adams, A. M.; Kaplan, N. A.; Wei, Z.; Brinton, J.; Monnier, C. S.; Enacopol, A.; Ramelot, T. A.; Jones, J. A. “In vivo production of psilocybin in E. coli.” Metabolic Engineering, 21 Sep. 2019, 56:111-119, abstract online at sciencedirect.com/science/article/abs/pii/S109671761930309X?via%3Dihub
US20210147888 Biosynthetic production of psilocybin and related intermediates in recombinant organisms
WO2021067626 Genetic engineering of fungi to modulate tryptamine expression
WO2021052989 Yeast cells and methods for production of tryptophan derivatives
WO2021086513 Methods for the production of psilocybin and intermediates or side products
CFR Title 21 Food and drugs, Part 1 to 1499, available online at ecfr.gov/current/title-21

1. A foodstuff made with a recombinant generally recognized as safe (GRAS) microbe that produces psilocybin, wherein said foodstuff has <10 mg or preferably <1 mg of psilocybin per serving of said foodstuff.

2. The foodstuff of claim 1, wherein said foodstuff has <250 μg of psilocybin per serving of said foodstuff.

3. The foodstuff of claim 1, wherein said foodstuff has <100 μg of psilocybin per serving of said foodstuff.

4. The foodstuff of claim 1, said foodstuff being an alcoholic beverage selected from beer, wine, cider, sake, or mead.

5. The foodstuff of claim 1, wherein said foodstuff is a beer and said beer has <250 μg of psilocybin per serving of beer.

6. A foodstuff made with a psilocybin producing recombinant microbe,
a) said microbe being of a species generally recognized as safe (GRAS) for human consumption;
b) said microbe expressing each of a PsiD gene, a PsiH gene, a PsiK gene, and a PsiM gene, each gene under the control of a weak promoter; and
a) wherein said foodstuff has <10 mg or preferably <1 mg of psilocybin per serving of said foodstuff.

7. The foodstuff of claim 6, wherein said foodstuff has <250 μg of psilocybin per serving of said foodstuff.

8. The foodstuff of claim 6, wherein said foodstuff has <100 μg of psilocybin per serving of said foodstuff.

9. The foodstuff of claim 6, said foodstuff being an alcoholic beverage selected from beer, wine, cider, sake, or mead.

10. The foodstuff of claim 6, wherein a psilocybin level varies by no more than ±5% from a stated psilocybin level on a packaging for said foodstuff.

11. The foodstuff of claim 6, wherein said foodstuff is a beer and said beer has <250 μg of psilocybin per serving of beer.
